(19) United States
(12) Patent Application Publication
(10) Pub. No.: US 2023/0115801 A1
(43) Pub. Date: Apr. 13, 2023

(54) Modular Remote Afterloader For Brachytherapy Delivery And Cartridge For Same

(71) Applicant: UNIVERSITY OF IOWA RESEARCH FOUNDATION, IOWA CITY, IA (US)

(72) Inventors: RYAN T. FLYNN, lowa City, IA (US); QUENTIN E. ADAMS, lowa City, IA (US)

(21) Appl. No.: 17/796,311

(22) PCT Filed: Jan. 29, 2021
(86) PCT No.: PCT/US2021/015628

§ 371 Date: Jul. 29, 2022

Related U.S. Application Data
(60) Provisional application No. 62/968,560, filed on Jan. 31, 2020.

(51) Int. Cl. A61N 5/10

(52) U.S. Cl. CPC A61N 5/1007; A61N 2005/101; A61N 2005/1008; A61N 2005/1009; A61N 2005/1094

(57) ABSTRACT

A replaceable cartridge can be used in a system for providing a radiation source for brachytherapy. The cartridge configured to be received into a base defining a receptacle. The cartridge can comprise a radiation source. A shield can be configured to receive the radiation source. A guidewire can be coupled to the radiation source. At least one motor can be configured to move the guidewire to thereby adjust the position of the radiation source.

### CROSS-REFERENCE TO RELATED APPLICATIONS

This application claims priority to and the benefit of the filing date of U.S. Provisional Application No. 62/968,560, filed Jan. 31, 2020, the entirety of which is hereby incorporated by reference herein.

### FIELD

This disclosure relates to brachytherapy delivery apparatuses and, in particular, to brachytherapy afterloaders having interchangeable cartridges.

### BACKGROUND

Brachytherapy is a radiation therapy method that involves inserting a radioactive material into the body of a patient and can be used to treat certain types of cancer. Afterloaders can be used in conjunction with a catheter inserted into the patient to control insertion of the radioactive material. Conventional afterloaders are heavy apparatuses that are kept at a treatment site. When the active source for the conventional afterloader is exhausted, the standard approach is for an engineer to travel to the treatment site to replace the active source and test the afterloader to confirm its operability. The afterloader can subsequently be used until the radioactivity of the active source is has decayed to a low enough level that active source replacement is needed, typically 90 days for a ¹⁹²Ir source. Because of the cost of replacing the active source in afterloaders, active source isotopes with longer half-lives such as ¹⁹²Ir (74-day half-life) or ⁶⁰Co (5.26 year half-life), are at an economic advantage regarding source replacement cost relative to a source with a shorter half-life such as ¹⁶⁹Yb (32-day half-life). However, ¹⁹²Ir and ⁶⁰Co have high average emitted photon energies of 380 keV and 1.25 MeV, and it can be desirable to use a source with lower average photon energy. ¹⁶⁹Yb has a 93 keV average emitted photon energy, providing the capability for intensity modulated brachytherapy (IMBT) delivery techniques such as rotating shield brachytherapy (RSBT) or dynamic modulated brachytherapy (DMBT), which could provide clinically superior radiation dose distributions to conventional approaches. However, the shorter half-life requires greater frequency of active source replacement, adding cost to the overall brachytherapy system.

### SUMMARY

Disclosed herein, in one aspect, is a replaceable cartridge that can be used in a system for providing a radiation source for brachytherapy. The cartridge can be configured to be received into a base defining a receptacle. The cartridge can comprise a radiation source. A shield can be configured to receive the radiation source. A guidewire can be coupled to the radiation source. At least one motor can be configured to move the guidewire to thereby adjust the position of the radiation source.

A system can comprise a cartridge comprising an active source and an afterloader base that is configured to receive the cartridge so that the cartridge is selectively removable and replaceable.

A method can comprise inserting a first cartridge into a receptacle of an afterloader base. The first cartridge can comprise an active source. The first cartridge can be removed from the receptacle of the afterloader base. A second cartridge can be inserted into the receptacle of the afterloader base.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIG. 1 is a schematic diagram of a side view of a modular afterloader system in accordance with embodiments disclosed herein.

FIG. 2 is a schematic diagram of a top view of a cartridge of the modular afterloader as in FIG. 1.

FIG. 3 is a schematic diagram of a cross sectional side view of a drive system of the cartridge of FIG. 2.

FIG. 4 is a schematic diagram of the cross sectional side view of FIG. 3 with the carriage in a second position.

FIG. 5A is a side view of a guide wire looped to receive a crimped tab. FIG. 5B is a side view of the guide wire of FIG. 5A with a tab crimped thereon.

FIG. 6 is a schematic diagram of an operating environment and a computing device that can be operable with the modular afterloader in accordance with embodiments disclosed herein.

FIG. 7A is a perspective view of a portion of a shield and a loading port/connector with a wedge section removed to show an interior of the shield. FIG. 7B is a perspective view of a portion of a shield and a loading port/connector with another wedge section removed to show an interior of the shield. FIG. 7C is a perspective view of a portion of a shield and a loading port/connector with a third wedge section removed to show an interior of the shield. FIG. 7D is a perspective view of a portion of a shield and a loading port/connector. FIG. 7E is a perspective view of a portion of a shield.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “a motor” includes one or more of such motors, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Intensity modulated brachytherapy (IMBT) can be clinically superior to conventional high-dose-rate brachytherapy (HDR-BT) due, in part, to the capability to deliver deliberately non-symmetric dose distributions that conform to tumors and avoid sensitive healthy tissues. An example of IMBT is rotating shield brachytherapy (RSBT), in which partially-shielded radiation sources with shields that rotate throughout treatment, enable improved tumor dose conformity, reduced normal tissue doses, or both. Conventional HDR-BT dose distributions, which are radially-symmetric about the implanted applicators and needles used for delivery, impose substantial geometric limitations on deliverable tumor dose conformity. RSBT has the potential to improve cervical cancer and prostate cancer therapy. Direction-modulated brachytherapy (DMBT) is another IMBT approach that could improve cervical cancer care. With DMBT, a multi-channel intracavitary applicator with a stationary central shield is used to improve tumor dose conformity and healthy tissue sparing relative to the conventional approach of using a tandem applicator without a central shield.

Delivering RSBT and DMBT dose distributions that are clinically equivalent or superior to those of conventional HDR-BT may require a lower-average-energy radiation source than conventional ¹⁹²Ir, which has an average emitted photon energy of 380 keV and a half-life of 74 days. This is because partial source shielding of ¹⁹²Ir within interstitial needles (2 mm diameter or less) can be ineffective due to high photon transmission, regardless of shielding material. For intracavitary RSBT and DMBT for cervical cancer, the small thickness of the radiation shield that can fit inside the applicator along with a ¹⁹²Ir HDR-BT source limits the tumor conformity benefits such that justifying replacement of the conventional, yet more invasive, combined intracavitary/interstitial brachytherapy approach is difficult.

A 27 Ci ¹⁶⁹Yb source (32 day half-life, 93 keV average energy), when unshielded, has a similar dose rate distribution to that of an unshielded 10 Ci ¹⁹²Ir source. When the ¹⁶⁹Yb source is partially shielded, potentially clinically superior RSBT and DMBT deliveries, relative to conventional HDR-BT, are possible. This is because of the lower average energy of the photons emitted by ¹⁶⁹Yb relative to conventional ¹⁹²Ir. Although a commercially available ¹⁶⁹Yb source exists, clinical usage of ¹⁶⁹Yb-based IMBT is impeded by three major problems: (i) applicators for delivering ¹⁶⁹Yb-based IMBT are commercially scarce, (ii)¹⁶⁹Yb sources are more expensive to generate than ¹⁹²Ir due to the high cost of precursor material such as 82%-enriched ¹⁶⁸Yb—Yb₂O₃, and (iii)¹⁶⁹Yb has a shorter half-life than ¹⁹²Ir (32 days vs 74 days), increasing replacement cost for vendors due to increased required replacement frequency.

To clinically justify the use of ¹⁶⁹Yb-based IMBT, prostate cancer and cervical cancer RSBT applicator designs have been developed and thoroughly evaluated with in silico studies. It has been demonstrated that a cervical cancer RSBT applicator can produce equivalent cervical cancer tumor dose conformity to conventional intracavitary/interstitial HDR-BT in half the treatment time without the invasive interstitial needles. International Patent Application No. PCT/US2019/052944, filed Dec. 13, 2019, which is hereby incorporated by reference in its entirety, discloses such a cervical cancer RSBT applicator. It has also been demonstrated that a prostate RSBT robotic delivery approach produced an average 30.8% prostate dose escalation relative conventional HDR-BT, without exceeding urethral and adjacent organ dose tolerances. International Patent Application No. PCT/US2020/023276, filed Mar. 18, 2020, which is hereby incorporated by reference herein in its entirety, discloses such a prostate RSBT robotic delivery system and method. These are clinically relevant advantages of ¹⁶⁹Yb RSBT that would not be possible with conventional ¹⁹²Ir-based HDR-BT.

The cost of ¹⁶⁹Yb sources has been addressed with a re-activatable ¹⁶⁹Yb source design that is expected to reduce the cost of ¹⁶⁹Yb source production by 75% relative to conventional ¹⁶⁹Yb sources, resulting in a precursor cost of only $14,532 per clinic-year in addition to that needed for conventional ¹⁹²Ir source production. International Patent Application No. PCT/US2020/017655, filed Feb. 11, 2020, which is hereby incorporated by reference herein in its entirety, discloses such a system and method for reactivating a source.

Overcoming the replacement cost of the source can include addressing the logistical challenge of providing clinics with replacement ¹⁶⁹Yb sources at least twice as often as conventional ¹⁹²Ir sources are currently delivered. The typical ¹⁹²Ir replacement model is for a vendor-employed engineer, funded through the service contract for the HDR-BT afterloader, to physically visit the clinical center four (4) times per year to replace the ¹⁹²Ir source, perform preventive maintenance on the afterloader, calibrate the afterloader, and return it to the center for continued clinical usage. The medical physicist then performs quality assurance tests on the remote afterloader, measures the air kerma strength (related to dose rate in water) of the ¹⁹²Ir source, updates the treatment planning system with the new air kerma strength value, and the authorized user(s) (physician(s)) can then proceed to treat patients. This process requires 0.5-1 full workday for the engineer and medical physicist, not including travel and lodging costs for the engineer, if necessary. The engineer and medical physicist must also coordinate the ¹⁹²Ir source replacement to occur at a mutually agreeable time when patient treatments are complete, which requires proactively scheduling no patient treatments during the time when the replacement and quality assurance is performed. Doubling the frequency of the source replacement process can dramatically increase service contract cost and would also be undesirable for end users due to the additional effort required to manage and quality assure the source replacements.

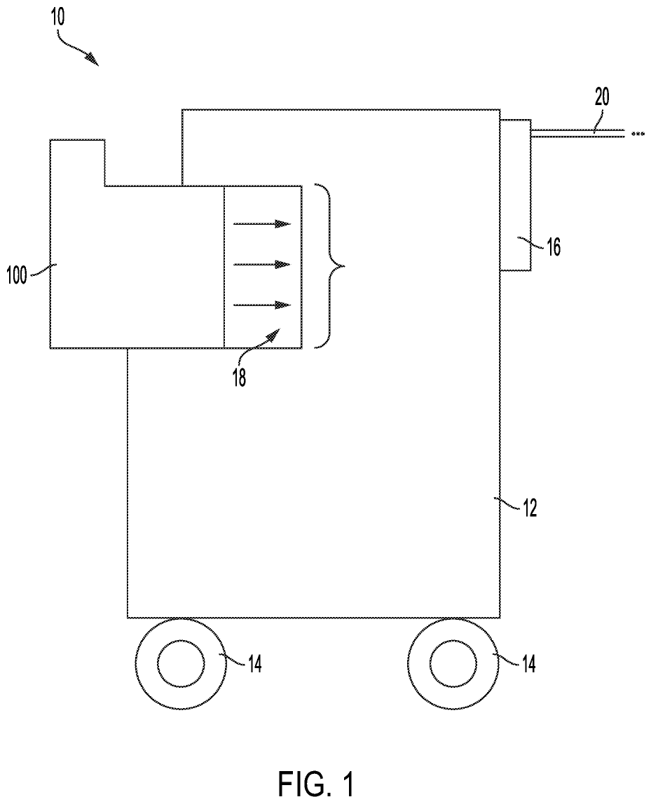

The disclosed apparatuses can make IMBT with isotopes with lower-average-photon energies than ¹⁹²Ir, such as ¹⁶⁹Yb, commercially possible. Disclosed herein in various aspects with reference to FIG. 1 is a modular remote afterloader (MRA) 10 that streamlines the source replacement and preventive maintenance processes relative to conventional afterloaders by reducing or eliminating the need to send an engineer onsite for those processes.

The MRA 10 can comprise a base 12 that is supported on wheels 14. The base 12 can comprise a channel selector 16 and a receiver/receptacle 18 that is configured to receive a cartridge 100. The base 12 can house the components for remote afterloader unit operation outside of that contained in the cartridge 100, optionally including the power source and control system for the cartridge. Accordingly, the base 12 can define a plurality of contacts that engage corresponding contacts of the cartridge 100 to provide electrical communication for power and control signals.

In some optional aspects, the base 12 can comprise a dummy source mechanism that can be used to test for successful source advancement through transfer tube(s) and applicator(s) prior to usage of the active source through the same pathway(s). As can be understood to one skilled in the art, the dummy source can comprise a piece of metal that has the same dimensions as the active source. The dummy source can be coupled to a dummy source wire. According to various aspects, the dummy source wire can be delivered through the applicator using conventional means. In further aspects, the dummy wire source can be controlled and manipulated using devices as further disclosed herein. Use of the dummy wire ensures there are no obstructions in the transfer tube(s) and applicator(s) that would cause a risk to the patient if present when the active source is used. In another aspect, the dummy wire and control mechanism can be located within the cartridge 100 and can optionally be controlled using a similar mechanism as the active guidewire, as further discussed herein.

One benefit of the disclosed system is the cartridge-based approach to performing rapid radiation source changes by on-site clinical staff. Rather than substituting just the guidewire and active radiation source as is done conventionally, a cartridge with a full-activity source (27-54 Ci of ¹⁶⁹Yb, for example), is substituted for the old cartridge with a “cool” source (10 Ci of ¹⁶⁹Yb, for example). This cartridge-swapping capability can be enabled by a compact mechanical guidewire drive assembly and compact shielded safe (referred to herein also as a “shield”), which provides both adequate shielding during cartridge transport, for example as a mailed Type A package in the United States, and personnel shielding for the staff and patients at the clinical site for entire time the source is onsite. Due to the lower average energy of radiation sources that could be used for IMBT (such as ¹⁶⁹Yb), the shielded safe can be, in some optional aspects, about 12.5 lb of lead (11.34 g/cm³) or about 7 lb of 3-D-printed tungsten (18.34 g/cm³), assuming 95% of maximum tungsten density (19.3 g/cm³) is achieved using a technique such as dynamic metal laser sintering (DMLS).

With conventional afterloaders, the source change process, which involves physically replacing the guidewire and radiation source, must be done on-site by specialized staff including an engineer typically employed by the afterloader vendor. A practical issue limiting the capability for an MRA approach for a 10-11 Ci ¹⁹²Ir afterloader is that the lead shielded safe needed to keep the dose rate outside of the afterloader within tolerance has a diameter of 21.2 cm and alone weighs nearly 125 pounds (e.g., Varian VariSource, U.S. Pat. No. 5,092,834). This volume and mass of shielding, if incorporated into a cartridge, is so bulky and heavy that the feasibility of giving clinical staff the responsibility of transporting and physically installing the cartridge is questionable.

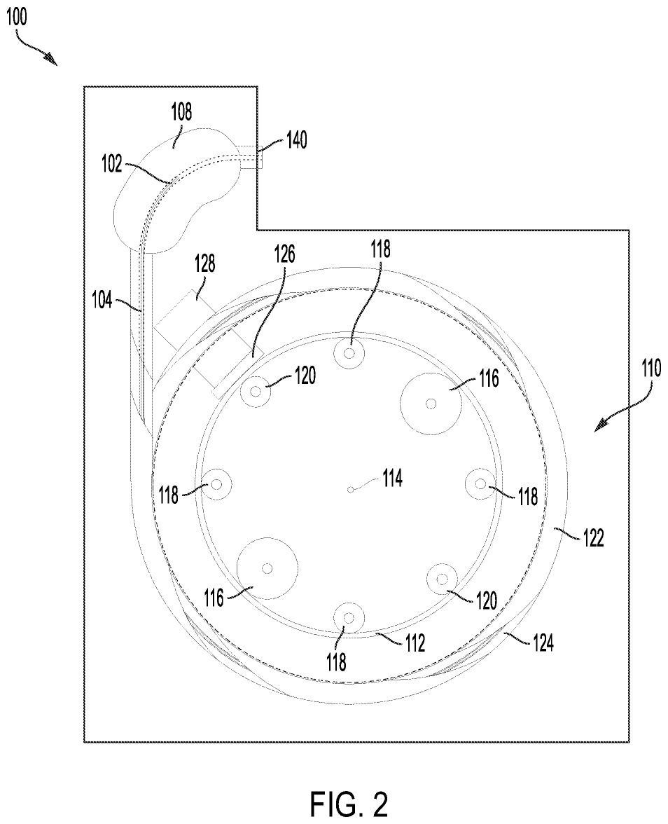

Referring to FIG. 2, the cartridge 100 can be configured to receive and deliver an active source 102 that is attached to a guidewire 104. The cartridge can comprise a housing 106 that houses a shield 108. The shield 108 can be configured to receive the active source 102 when the active source is in a retracted position. When the active source 102 is disposed within the shield 108, the shield can limit the radiation emitted from the cartridge to within a predetermined range. In some aspects, the predetermined range can be set by regulatory agencies.

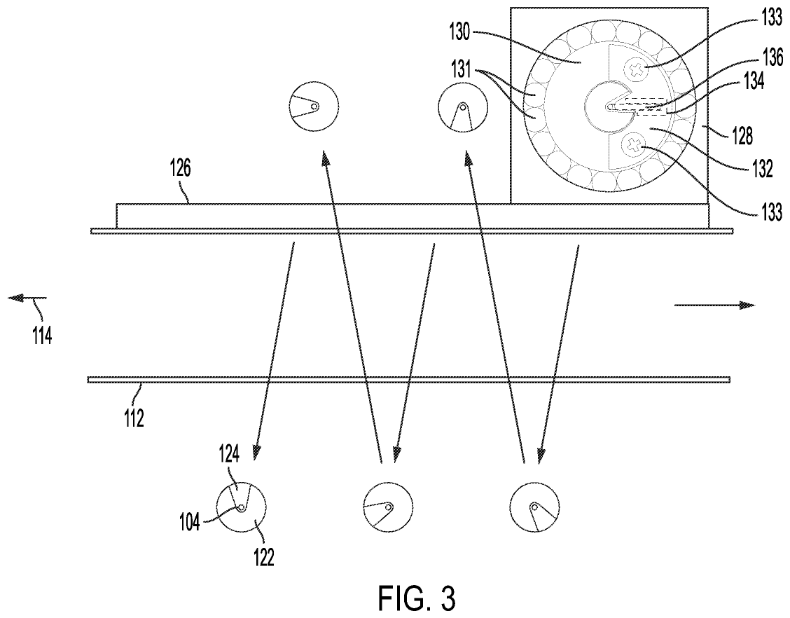

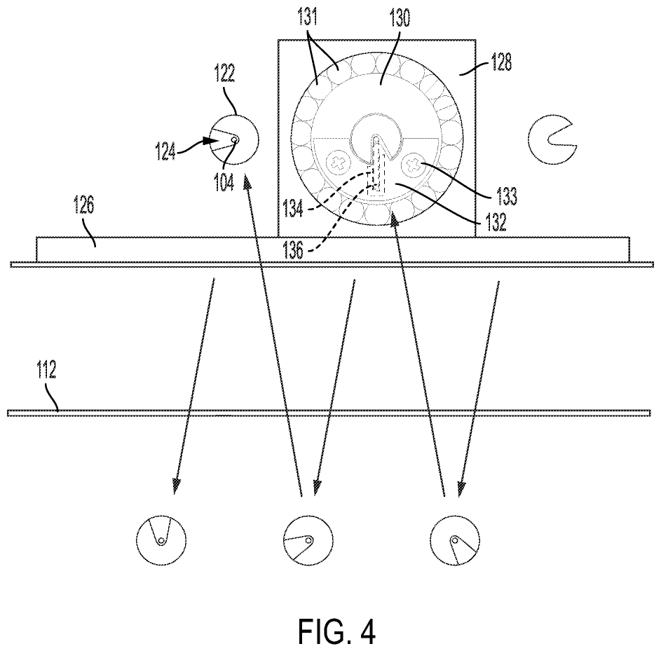

Referring also to FIGS. 3 and 4, a guidewire movement assembly 110 can be configured to advance the guidewire 104 to thereby move the active source from the cartridge, through the base, through an applicator tube, and into a catheter within a patient. The guidewire movement assembly 110 can comprise a barrel 112 that is disposed within the housing 106 and rotatable about a rotational axis 114. The barrel 112 can be rotationally supported on rollers 118 (e.g., four rollers 118). Optionally, the rollers 118 can support the barrel 112 from the interior of the barrel. At least one motor 116 (e.g., two motors) can engage the barrel 112 so that rotation of the motor(s) causes rotation of the barrel. At least one encoder 120 (e.g., two encoders) can be in communication with the barrel in order to measure the rotation angle of the barrel. The plurality of motors 116 and encoders 120 can provide redundancy so that failure of one does not prohibit operability of the cartridge 100 and, thus, the MRA 10. It is contemplated that the encoders 120 disclosed herein can be a rotary encoder, an absolute encoder, and/or an incremental encoder as is known in the art. In use, it is contemplated that the encoders 120 can be configured to provide an output that is indicative of an angular or rotary position (or a change in angular or rotary position) of an object (e.g., the barrel).

A guide tube 122 can extend within the housing along a spiral (e.g., helical) path with the barrel 112 extending therethrough. The guide tube 122 can be coupled to the housing 106 so that as the barrel rotates therein, the guide tube can remain fixed. The guide tube 122 can define a helical groove 124 that extends at least to the center of the guide tube with respect to a cross section that is perpendicular to the spiral path of the guide tube. In some optional aspects, the helical groove 124 can extend beyond the center of the guide tube by at least the radius of the guide wire 104. The helical groove 124 can optionally subtend an angle of about thirty degrees (optionally, an angle of about 20 to about 40 degrees) along the length of the helical guide tube. The guide tube 122 can receive the guidewire 104 within the helical groove 124 so that the guidewire 104 extends along the centerline of the guide tube. It is contemplated that a forty-five degree groove rotation (optionally, a groove rotation of about 35 to about 55 degrees) can prevent the guidewire 104 from undesirably escaping the groove, particularly over a short distance (e.g., 2.5 cm). Thus, it is contemplated that the helical groove 124 can have a pitch of about twenty cm (optionally, from about 15 cm to 25 cm), along the length of the guidewire 104, per rotation. Optionally, the guidewire 104 can comprise a material such as stainless steel, titanium, or NiTi and can have a diameter of about 0.9 mm (optionally, ranging from about 0.7 mm to about 1.1 mm). Accordingly, the guidewire 104 can have a degree of rigidity and, thus, stay within the groove when the guidewire is pushed from an end opposite the active source.

A carriage track 126 can couple to the barrel 112 so that the carriage track rotates with the barrel about the rotational axis 114 and can extend along the barrel in a direction that is parallel to the rotational axis of the barrel. A carriage 128 can be movable along the carriage track 126. A rotator 130 can be rotatably disposed within the carriage 128 and supported by ball bearings 131. The rotator 130 can receive the guide tube 122 therethrough so that the carriage 128 and rotator 130 follow the helical path of the guide tube 122. The rotator 130 can rotate about the tangent of guide tube 122 and, thus, an axis that is tangent to the direction of movement of the carriage.

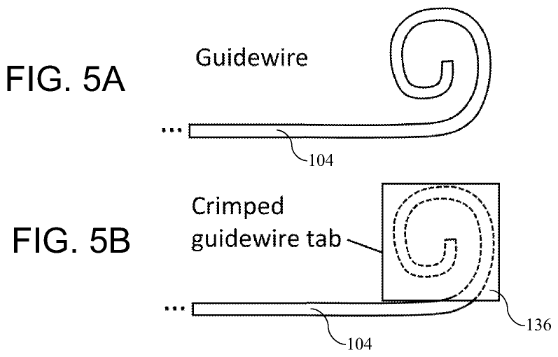

The rotator 130 can couple to the guidewire 104. For example, the rotator 130 can comprise a detachable locking plate 132 that defines a tab guide 134. Referring also to FIGS. 5A and 5B, a tab 136 can attach to the guidewire 104. For example, a portion of the guidewire 104 can form at least a portion of a loop, and the tab 136 can be crimped to the loop of the guidewire so that it extends laterally from the axis of the wire. The tab 136 can optionally comprise stainless steel, titanium, aluminum, or the like. The tab 136 can couple to the tab guide 134. In this way, the guidewire movement assembly 110 can couple to the guidewire 104.

As the motors 116 cause the barrel 112 to rotate, the coupling between the carriage track 126 and the barrel drives the carriage 128 (and, thus, the rotator 130) along the guide tube 122. The coupling between the rotator 130 and the guidewire 104 pushes the guidewire through the guide tube 122 and out of the cartridge 100. Because of the helical shape of the groove 124, the guidewire 104 can remain within the groove as the force is applied from the end opposite the active source. The rotation of the rotator enables the tab to remain engaged with the tab guide as the coupled tab and tab groove slide along the helical groove. In this way, the guidewire movement assembly 110 can advance the active source into the transfer tube and the delivery applicator. It is contemplated that the encoders can measure the angular displacement of the barrel, which can correspond to the distance of travel of the guidewire and active source. For example, a single revolution can correspond to the length of the guide tube along its helical path for a single turn. Thus, it is contemplated that the encoders can be in communication with a controller (or any suitable computing device) that can calculate the displacement of the active sources from its retracted position or the change of position of the active source. In various aspects, the controller can be disposed within the base, the cartridge, or a station that is remote from the base and cartridge.

In the reverse, to retract the guidewire 104 and active source 102, the motors 116 can run in the reverse direction to pull the guidewire 104 back into the guide tube until the active source 102 is disposed within the shield 108.

The guide tube 122 can optionally have a diameter of about 1 cm (optionally, from about 0.8 cm to about 1.2 cm). In some optional aspects, the barrel 112 can have a length of about 13 centimeters (optionally, from about 10 cm to about 15 cm) relative to the longitudinal axis 114. The barrel can have a diameter of about 14 cm (optionally, from about 12 to about 16 cm). Optionally, the guide tube 122 can be spaced radially from the outer surface of the barrel 112 by about 2.5 cm (optionally, by about 2.2 cm to about 2.8 cm). The carriage track 126 can extend radially from the outer surfaced of the barrel 112 by about 0.5 cm (optionally, from about 0.3 cm to about 0.7 cm). Adjacent coils of the helical guide tube 122 can be spaced axially by about 3 cm (optionally, from about 2 cm to about 4 cm).

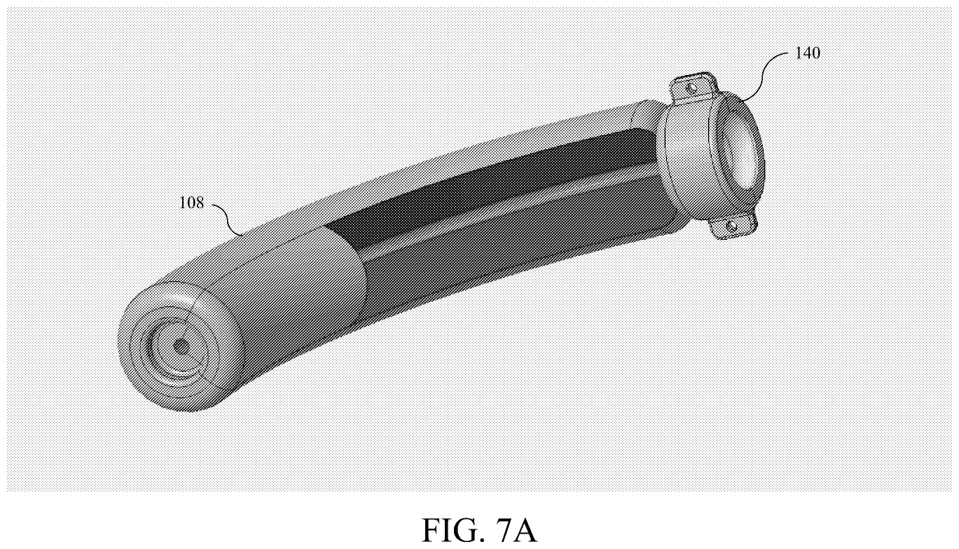

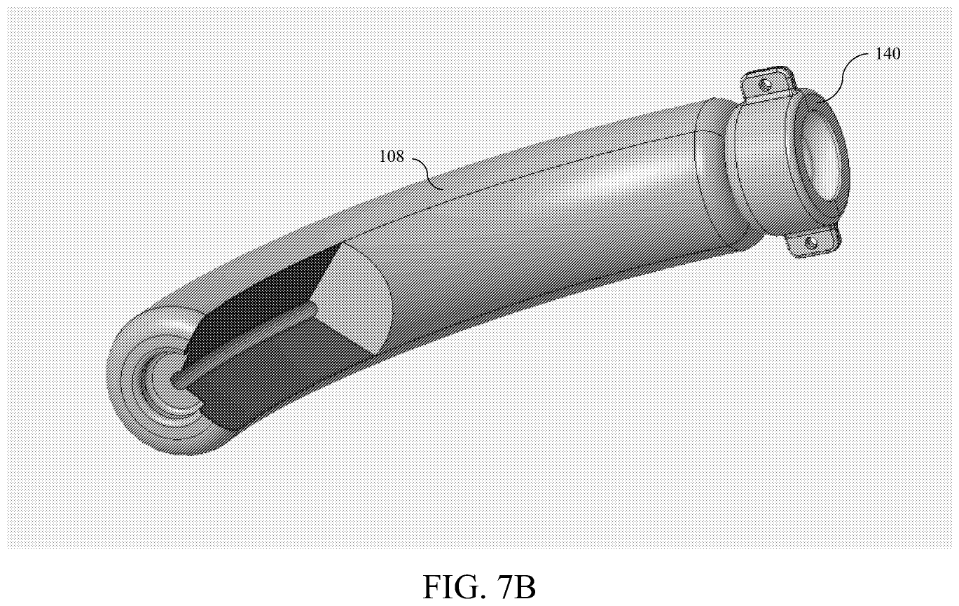

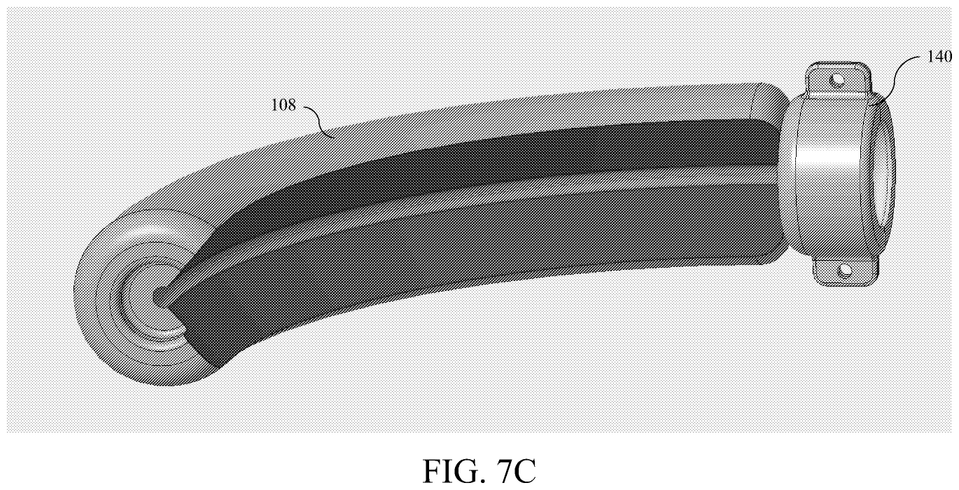

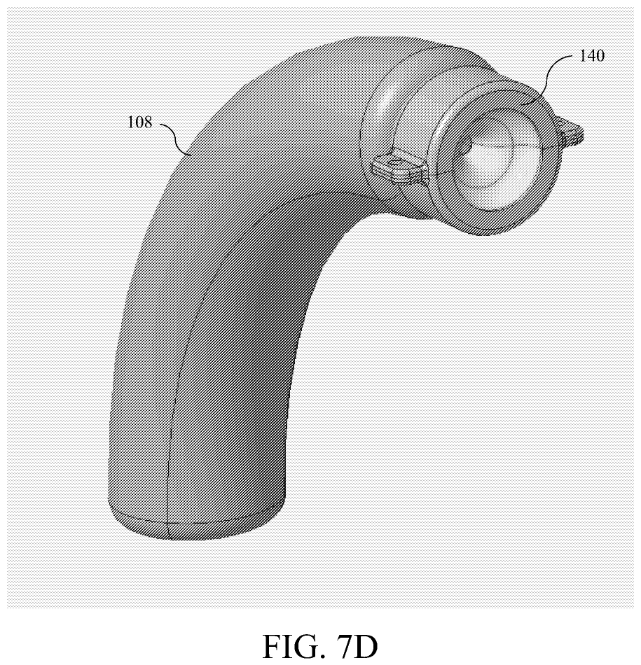

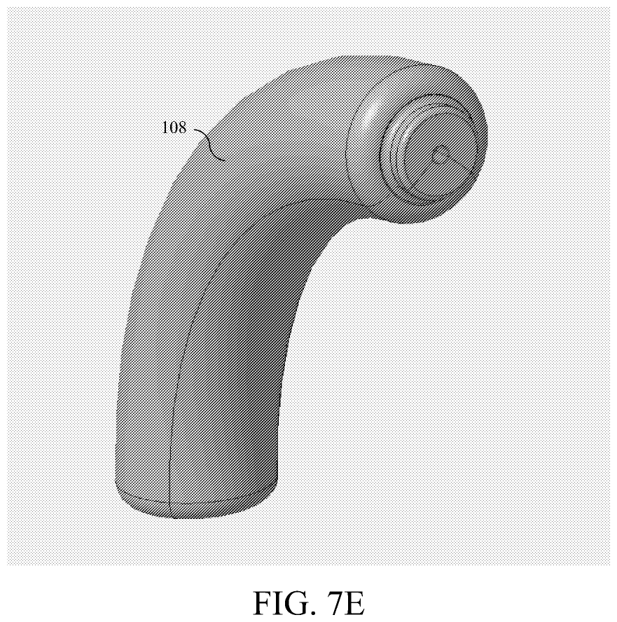

Referring to FIGS. 7A-E, a loading port/connector 140 can couple to an end of the shield 108. When the cartridge 100 is coupled to the base 12, the loading port/connector 140 can align a tube leading to channels of the base. That is, the loading port/connector 140 can be configured to mate with the base 12 so that as the active source exits the cartridge, the active source is guided into the tube leading to channels of the base. Transfer tubes 20 for radiation delivery can be connected to the respective channels.

Optionally, the loading port/connector 140 and a portion of the shielded safe 108 (FIGS. 7A-7E), or the entirety of the shielded safe, can be removed to provide access to the helical guide tube and the guidewire tab. After the guidewire tab is in the helical guide tube and secured in the carriage, the shielded safe and the loading port/connector can be put back in place within the cartridge. In some aspects, the shielded safe 108, or the removable portion of the shielded safe, can be secured into place in a wedged fashion (e.g., with a slight interference fit). The rotating barrel can be engaged, thereby pulling the source inside the shielded safe.

The shield 108 for the source can have a low enough mass to enable easy manual cartridge portability. The shield thickness, which corresponds to shield mass, needed to provide the same broad beam transmission for 27 Ci of ¹⁶⁹Yb as the 10.6 cm radius of lead used in the Varian VariSource system (U.S. Pat. No. 5,092,834) for 10 Ci of ¹⁹²Ir, can be determined using the parameters from the literature in Table 1.

TABLE 1
Shield properties for the photons emitted by ¹⁹²Ir and ¹⁶⁹Yb. Tenth-value-layers
are based on realistic, broad-beam-geometry conditions in which build-up inside
the shielding material and scatter are accounted for. Shield properties are
shield thickness, x, and transmission in broad-beam conditions, B.

| | | | | | | Shield Properties | | |
|---|---|---|---|---|---|---|---|---|
| | | Tenth-value-layer [cm] | | | | x | B | |
| Isotope | Material | 1 | 2 | 3 | 4+ | [cm] | [unitless] | Source |
| ¹⁹²Ir | Lead | 1.2 | 1.5 | 1.5 | 1.5 | 10.6 | 5.4 × 10⁻⁸ | Canadian Nuclear |
| | | | | | | | | Safety Commission |
| ¹⁶⁹Yb | Lead | 0.16 | 0.46 | 0.58 | 0.6 | 3.16 | 5.4 × 10⁻⁸ | Granero et al Monte |
| | | | | | | | | Carlo calculations |

Broad beam transmission data for ¹⁶⁹Yb photons for tungsten shielding are unavailable in the literature. An estimate can be calculated for the tungsten shield thickness, x_{W} [cm], needed to obtain a broad beam transmission, B [unitless], of 5.4×10⁻⁸, consistent with the Varian VariSource system. The estimate can be performed by scaling the lead thickness, x_{Pb} [cm], needed to achieve B=5.4×10⁻⁸, by the ratio of analytically calculated narrow-beam-geometry thicknesses for tungsten, t_{W} [cm], and lead, t_{Pb} [cm] as follows:

$${x}_{W} = {x}_{P b} \frac{{t}_{W}}{{t}_{Pb}} .$$

The t_{W} and t_{Pb} values were obtained by iteratively determining the respective t-value that resulted in a narrow-beam transmission, T [unitless], of:

$$T = \frac{{\sum }_{i = 1}^{N} {Ψ}_{i} {e}^{- {μ}_{i} t}}{{\sum }_{i = 1}^{N} {Ψ}_{i}} = 5.4 \times 1 {0}^{- 8} , where {μ}_{i} = \frac{{μ}_{e n}}{ρ} ( h {v}_{i} ) {ρ}_{s} [ {cm}^{- 1} ]$$

is the attenuation coefficient of the shielding material for the photon energy

$$h {v}_{i} ( i = 1 , … , N ) [ keV ] , \frac{{μ}_{e n}}{ρ} ( h {v}_{i} ) [ {cm}^{2} {g}^{- 1} ]$$

is the mass energy absorption coefficient for the shielding material for photon energy index i, ρₛ is the density of the shielding material [g cm⁻³], and Ψᵢ is the energy fluence [keV cm⁻²] for photon energy index i. The calculated values are t_{Pb}=5.39 cm, t_{W}=3.93 cm, and x_{W}=2.31 cm. As a sensitivity check for the method of attenuation coefficient calculation, the mass attenuation coefficient,

$$\frac{μ}{ρ} ( h {v}_{i} ) [ {cm}^{2} {g}^{- 1} ] ,$$

can be used in the calculation of the equation for determining T, rather than

$$\frac{{μ}_{e n}}{ρ} ( h {v}_{i} ) ,$$

and the calculated values are t_{Pb}=3.28 cm, t_{W}=2.39 cm, and x_{W}=2.30 cm. The x_{W}-result is thus insensitive to the choice of attenuation coefficient calculation, and 2.31 cm can be a conservative thickness of 3-D printed tungsten shielding needed to achieve the same dose rate outside the remote afterloader shielding for 27 Ci of ¹⁶⁹Yb as 10 Ci of ¹⁹²Ir. Enabling ¹⁶⁹Yb activities of up to 54 Ci to be transported and delivered can be achieved by adding another half-value-layer of shielding, which can be another 0.6 cm/log₂(10)=0.18 cm of lead or (0.18 cm)(39.3/53.9)=0.13 mm of 3-D printed tungsten. The total lead shield thickness can thus be 3.34 cm and the total tungsten shield thickness can be 2.44 cm.

The shielded safe used for the cartridge can be approximated as a quarter-torus with a hollow pathway along the torus axis with a radius of 0.2 cm, which can provide adequate space for a guidewire tube through which the source can travel. The mass of the shielded safe, Mₛ [kg], can be calculated be scaling the toroidal volume by the density of the shield as:

$${M}_{s} = \frac{2 {π}^{2}}{1 {0}^{3}} R ( {r}_{1}^{2} - {r}_{2}^{2} ) \frac{Δ θ}{360 °} {ρ}_{s} ,$$

where R [cm] is the distance between the torus origin (geometric center) and the center of an axial (circular) cross section of the torus, r₁ [cm] is the outer radius of the axial torus cross section, r₂ [cm] is the radius of the hollow toroidal pathway through the shield, and Δθ [degrees] is the angular arc subtended by the torus. In optional exemplary embodiments, the shield can comprise lead or tungsten, with R=5 cm, r₂=0.2 cm, and AB=90°. For both exemplary shields, r₁=r₂+Δr, where Δr is the shielding thickness needed to support 54 Ci of ¹⁶⁹Yb, therefore r₁=3.54 cm for lead and r₁=2.64 cm for tungsten. Using the densities for lead and 3-D printed tungsten, calculated shield masses are 5.653 kg (12.44 lb) for lead and 3.130 kg (6.89 lb) for tungsten. It is possible that shielded safe mass can be reduced through geometric optimization by rounding-off the edges to create half-spherical boundaries at the end where the source enters and exits. Accordingly, the mass of the shield can be further reduced. As of Dec. 15, 2019, the price of lead was $0.88 per pound, whereas the price of pure tungsten powder needed for use in additive manufacturing techniques, which would be expected to be necessary to generate the shield in an appropriate shape and density for clinical use was $6,254 per pound. Other high-density metals such as iridium, uranium, and osmium, can optionally be used, but the price per pound for those metals was even higher than for tungsten for this purpose. Lead can therefore be a cost-effective material for use in the cartridge shielded safe.

### Methods for Changing the Active Source

Radiation source and guidewire loading in a cartridge can be achieved in a two-part process. In this description, it will be assumed that an empty cartridge is being loaded with an active source, thus a depleted active source has already been removed. A fresh active source can be in a shielded container that attenuates the radiation emitted by the source sufficiently to protect the personnel involved. The new guidewire can protrude from the shielded container. In a first loading stage, the new guidewire can be fed into the carriage, the guidewire tab can be locked in place in the carriage, and the carriage closed in preparation for the source to be pulled in by rotating the barrel. The personnel performing the first loading stage can do so in physical contact with the cartridge and new guidewire, as they will not be exposed to the radiation from the active source to a significant degree during that process. In a second loading stage, the barrel can be activated, the guidewire can be pulled such that the active source is removed from its shielded container pulled into the cartridge, and the cartridge containing the source can then come to a rest in the shielded safe. The personnel performing the second stage can do so remotely since the active source will be briefly exposed, thus under optimal conditions, the cartridge, guidewire, and source container should be inside a shielded room or shielded box to protect the personnel from exposure to radiation from the source. A receiver for the cartridge that provides the necessary electrical and electronic connections to operate the cartridge remotely can be required inside the shielded room or box.

A detailed description of stage one of a source change, in optional aspects, proceeds as follows. First, the personnel rotate the barrel until the carriage is at the 9 o'clock position, as shown in FIG. 2, on the final coil wrapping prior to the loading port, getting the carriage close to the loading port in preparation to receive a new guidewire and radiation source. The locking plate screws 133 (FIG. 4) can be removed from the carriage and the locking plate removed as well, so the carriage is ready to receive a guidewire tab. The active source can be inside a shielded container and the guidewire can be freely accessible. The loading port/connector can be removed along with a removable section of the shielded safe (FIG. 2), enabling the guidewire tab (FIG. 5B) to be placed in the distal helical guide tube, oriented in alignment with the helical keyway. The guidewire can be manually or automatically pushed through the helical tube for a distance of approximately 10 cm, rotating in accordance with the helix formed by the keyway until the guidewire tab reaches the carriage. Once the guidewire tab is inside the carriage, the guidewire tab can be mechanically prevented from proceeding any further down the helical guide tube. The locking plate and corresponding screws can be re-installed on the carriage, locking the guidewire tab in place. The shielded safe and loading port/connector can be re-installed, as they are designed to be installed around a guidewire. This can lock the guidewire in place, completing the lateral wall of a tube between the helical guide tube and distal exit of the loading port/connector. In the second loading stage, the shielded cartridge loading container or the door to the shielded room can then be closed, and the control system can be engaged to rotate the barrel, counter-clockwise from the view shown in FIG. 2, until the guidewire is pulled inside the helical guide tube and the source is in the center of the shielded safe.

Source/guidewire removal can be accomplished by reversing the loading process. This process can be repeated many times without the replacement of the major functional components of the system.

In further optional aspects, the guidewire 104, when retracted, can be spooled up within a coiled tube or wrapped around a spool. The guidewire 104 can extend between a pair of drive wheels (or, optionally, a single wheel and a biasing surface) that can bias against opposing sides of the guide wire. The wheel(s) can rotate in respective first rotational directions so that the vector of the movement of the portion of each wheel in engagement with the guidewire is in the same linear direction. In this way, rotation of the wheels can impart linear motion on the guidewire to drive the guidewire in the first linear direction and draw the spooled portion of the guidewire from the coiled tube or spool. The wheel(s) can rotate in respective second rotational directions to retract the guidewire into the coiled tube or spool. A similar guidewire drive system is disclosed in U.S. Pat. No. 5,800,333, issued Sep. 1, 1998 to Samuel Lipre, the entire disclosure of which is hereby incorporated by reference herein.

### Applications

Business and clinical workflows for the combined re-activatable radiation source, RSBT applicators, and the MRA are further disclosed herein. In the business workflow, a first business entity/operation (e.g., a licensee) can manage ¹⁶⁹Yb source installation, source removal, and preventive maintenance for the cartridges. The first business entity/operation can send and receive the radiation sources to and from the reactor, which can receive and activate or reactivate the sources and send them back to the first business entity/operation for installation in the cartridges. An end user (e.g., a Clinic) can receive a cartridge, install it in the MRA, run a simple quality assurance routine, and treat patients, for example using the RSBT applicators, for the appropriate amount of time for the source, which is 39 days in this example, corresponding to a 27 Ci source on delivery to the clinic. An advantage of the MRA is that the first business entity/operation does not need to send an engineer to the Clinic to install the radiation source. When the dose rate of the radiation source is below the desired level, the cool (old) cartridge would be removed and mailed back to the first business entity/operation, starting the process over. The Clinic does not have to mail the cool cartridge back to the first business entity/operation until after the hot (new) cartridge and the overall MRA has passed all quality assurance tests.

### Computing Device

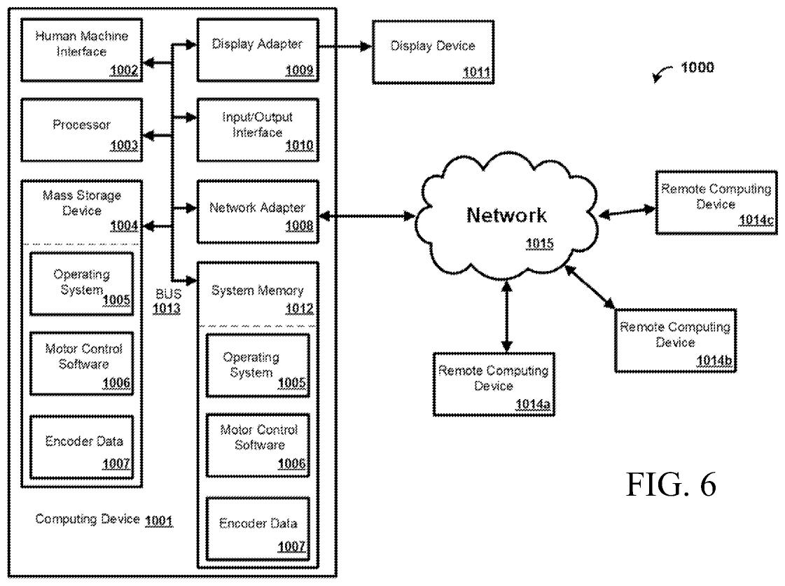

FIG. 6 shows a system 1000 including a computing device 1001 for use with the MRA 10. In exemplary aspects, the computing device 1001 can be, for example, a personal computer, computing station (e.g., workstation), a portable computer, such as laptop computers, or a server. In further aspects, the computing device 1001 can be a programmable logic controller (PLC). In various aspects, the MRA 10 can utilize a plurality of computing devices that are consistent with the described architecture and operation of the computing device 1001. For example, the cartridge can comprise a first computing device; the base can comprise a second computing device; and a third computing device, which can be remote from the MRA 10, can enable a user to provide inputs to and control the MRA 10. In other aspects, one controller may be configured to control one or more of the cartridge, the base, and the remote computing device. For example, in some aspects, the base can comprise an input/output (I/O) port that is in communication with a remote computer that an operator can interface with. The receptacle of the base can further enable communication so that the cartridge can interface with the remote computer through the I/O port of the base. In this way, a single computing device can provide for operator interfacing, all operation of the base the cartridge, and further data processing. In further aspects, the base can comprise a controller that can control operation of both the base and the cartridge. In still further aspects, the cartridge can comprise a controller that can control operation of both the base and the cartridge.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as encoder data 1007 and/or program modules such as operating system 1005 and motor control software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and the motor control software 1006 may be stored on the mass storage device 1004. One or more of the operating system 1005 and the motor control software 1006 (or some combination thereof) may comprise program modules and the motor control software 1006. The encoder data 1007 may also be stored on the mass storage device 1004. The encoder data 1007 may be stored in any of one or more databases known in the art. The databases may be centralized or distributed across multiple locations within the network 1015.

A user may enter commands and information into the computing device 1001 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices may be connected to the one or more processors 1003 via a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 via an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 via Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made via a network 1015, such as a local area network (LAN) and/or a general wide area network (WAN). Such network connections may be through a network adapter 1008. A network adapter 1008 may be implemented in both wired and wireless environments. Such networking environments are conventional and commonplace in dwellings, offices, enterprise-wide computer networks, intranets, and the Internet.

Application programs and other executable program components such as the operating system 1005 are shown herein as discrete blocks, although it is recognized that such programs and components may reside at various times in different storage components of the computing device 1001, and are executed by the one or more processors 1003 of the computing device 1001. An implementation of the motor control software 1006 may be stored on or sent across some form of computer readable media. Any of the disclosed methods may be performed by processor-executable instructions embodied on computer readable media.

The computing device 1001 (or one of such computing devices) may be configured to operate the at least one motor to move the active source. Similarly, the computing device 1001 may be configured to control the position of the dummy wire (e.g., by operating a motor of a similar drive assembly to that of the active source or another drive assembly for extending and retracting the dummy wire. The MRA 10 can comprise at least one radiation detector. The computing device can receive signals from the radiation detector and, based on signals from the radiation detector, determine whether the active source has been extended or retracted. For example, the radiation detector can measure radiation in the room. The radiation measurements can be compared (e.g., via a controller) to ambient radiation in the room. Thus, a radiation measurement that is higher than the ambient radiation in the room can indicate that the source is not within the shield 108.

The controller (e.g., computing device 1001) can be configured to determine a current measurement through each of the motors of the cartridge and/or the base. Based on the current measurements, the controller can determine if the motor current has exceeded a first threshold. For example, the motor current exceeding a threshold can indicate that the guidewire or dummy wire is jammed (e.g., from the tab guide breaking or the rotator component getting stuck). In some aspects, the threshold for a jammed motor can be different depending on the direction of the motor rotation. Accordingly, the motor can have a first threshold for the first direction of rotation and a second threshold for the second direction of rotation.

In some aspects, the controller can determine an error in operation. For example, the controller can determine that the guidewire is jammed. The controller can, in response, automatically retract the guidewire so that the active source is repositioned into the shield. As another example, if there is a motor malfunction, the barrel may not rotate as quickly as expected, causing the guidewire to travel through the transfer tube too slowly and causing excess dose to the patient due to slow source travel. Such a situation could trigger a controller error. In further aspects, an error can be triggered if an output of a first encoder 120 does not match an output of a second encoder 120.

The same or a different controller can be configured to determine whether the dummy wire is advancing properly through the applicator. For example, a motor current exceeding a threshold can indicate that the dummy wire is jammed. In further aspects, the encoders indicating a lack of rotation of the barrel, or a load cell indicating a threshold back-force can indicate that the dummy wire is jammed.

### Exemplary Aspects

In view of the described device, systems, and methods and variations thereof, herein below are certain more particularly described aspects of the invention. These particularly recited aspects should not, however, be interpreted to have any limiting effect on any different claims containing different or more general teachings described herein, or that the “particular” aspects are somehow limited in some way other than the inherent meanings of the language literally used therein.

Aspect 1: A cartridge that is configured to be received into a base defining a receptacle, the cartridge comprising: a radiation source; a shield that is configured to receive the radiation source; a guidewire coupled to the radiation source; and at least one motor that is configured to move the guidewire to thereby adjust the position of the radiation source.

Aspect 2: The cartridge of aspect 1, further comprising: a housing; a barrel that is coupled to the at least one motor so that rotation of the at least one motor causes the barrel to rotate within the housing about a rotational axis; a guide tube that is wrapped around the barrel and that is coupled to the housing so that it remains fixed within the housing as the barrel rotates; a carriage track that is coupled to the barrel so that the carriage track rotates around the rotational axis as the barrel rotates, wherein the carriage track extends along the barrel in a direction that is parallel to the rotational axis; a carriage that is movable along the carriage track and simultaneously along the guide tube; and a rotator that is housed within the carriage so that the rotator is rotatable about an axis that is tangent to a direction of movement of the carriage, wherein the rotator is coupled to the guidewire so that as the carriage moves, the guidewire advances through the guide tube.

Aspect 3: The cartridge of aspect 1 or aspect 2, wherein the at least one motor comprises two motors.

Aspect 4: The cartridge of any one of the preceding aspects, wherein the guide tube defines a helical notch.

Aspect 5: The cartridge of any one of the preceding aspects, wherein the shield has a quarter toroid shape.

Aspect 6: The cartridge of any one of the preceding aspects, further comprising at least one encoder that is configured to measure an angular displacement of the barrel.

Aspect 7: The cartridge of any one of the preceding aspects, further comprising a tab that extends laterally from the guidewire.

Aspect 8: The cartridge of any one of the preceding aspects, further comprising a plurality of rollers that rotatably support an interior of the barrel.

Aspect 9: The cartridge of any one of the preceding aspects, further comprising at least one of a power source or a controller that is configured to operate the at least one motor.

Aspect 10: The cartridge of any one of the preceding aspects, further comprising a dummy source wire.

Aspect 11: The cartridge of any one of the preceding aspects, wherein at least a portion of the shield is removable and replaceable.

Aspect 12: The cartridge of any one of the preceding aspects, further comprising a loading port, wherein at least a portion of the loading port is removable and replaceable.

Aspect 13: The cartridge of any one of the preceding aspects, wherein the shield is a first shield, wherein the cartridge is configured to receive a second shield in place of the first shield, wherein the second shield differs from the first shield in at least one of size or shape.

Aspect 14: The cartridge of any one of aspects 2-13, wherein the guide tube is a first guide tube, wherein the cartridge is configured to receive a second guide tube in place of the first guide tube, wherein the second guide tube differs from the first guide tube in pitch.

Aspect 15: The cartridge of aspect 10, further comprising a drive and control system that is configured to extend the dummy wire from the cartridge.

Aspect 16: A system comprising: a cartridge as in any one of the preceding claims, and a base comprising: a receptacle that is configured to receive the cartridge, wherein the receptacle defines a plurality of contacts for providing power and control signals to the cartridge; a controller that is configured to communicate with the cartridge via the plurality of contacts.

Aspect 17: The system of aspect 16, wherein the base further comprises a dummy source wire.

Aspect 18: The cartridge of claim 17 further comprising a drive and control system that is configured to extend the dummy wire from the cartridge.

Aspect 19: The system of any one of aspects 16-18, wherein the base comprises a channel selector.

Aspect 20: The system of aspect 19, further comprising a plurality of transfer tubes each associated with a respective channel, wherein the channel selector is configured to select between the respective channels.

Aspect 21: The system of any one of aspects 16-20, further comprising at least one radiation detector.

Aspect 22: The system of aspect 21, further comprising a controller that is operable: to receive signals from the at least one radiation detector; and determine, based on the signals received from the at least one radiation detector, if the radiation source has been extended or retracted from the cartridge.

Aspect 23: The system of aspect 21 or aspect 22, wherein the at least one radiation detector is in the cartridge.

Aspect 24: The system of aspect 21 or aspect 22, wherein the at least one radiation detector is in the base.

Aspect 25: The system of any one of aspects 16-24, further comprising a controller that is configured to determine a current measurement through the at least one motor and, based on the current measurement, if the motor current has exceeded a first threshold.

Aspect 26: The system of aspect 25, wherein the controller is configured to determine whether the current has exceeded a second threshold, wherein the first threshold is associated with rotation in a first direction, and the second threshold is associated with rotation in a second direction that is opposite the first direction.

Aspect 27: The system of any one of aspects 16-26, further comprising a battery power source which can be run independently from the primary power source in the event of an irreversible power loss.

Aspect 28: The system of any one of aspects 16-27, further comprising a controller that is configured to detect an error in operation.

Aspect 29: The system of aspect 28, wherein the controller is configured to cause the cartridge to retract the active source in response to detecting the error in operation.

Aspect 30: The system of any one of aspects 16-28, further comprising an input device that is configured to receive an input from a user.

Aspect 31: The system of claim 30, wherein the system is configured to extend and retract the active source based on the input from the user.

Aspect 32: The system of aspect 30 or aspect 31, wherein the input device is remote from the base and the cartridge.

Aspect 33: A system comprising: a cartridge comprising an active source; and an afterloader base that is configured to receive the cartridge so that the cartridge is selectively removable and replaceable.

Aspect 34: A method comprising: inserting a first cartridge into a receptacle of an afterloader base, wherein the first cartridge comprises an active source.

Aspect 35: The method of aspect 34, further comprising: removing the first cartridge from the receptacle of the afterloader base; and inserting a second cartridge into the receptacle of the afterloader base.

Those skilled in the art will recognize, or be able to ascertain using no more than routine experimentation, many equivalents to the specific embodiments of the method and compositions described herein. Such equivalents are intended to be encompassed by the following claims.

1. A cartridge that is configured to be received into a base defining a receptacle, the cartridge comprising:
a radiation source;
a shield that is configured to receive the radiation source;
a guidewire coupled to the radiation source; and
at least one motor that is configured to move the guidewire to thereby adjust the position of the radiation source.

2. The cartridge of claim 1, further comprising:
a housing;
a barrel that is coupled to the at least one motor so that rotation of the at least one motor causes the barrel to rotate within the housing about a rotational axis;
a guide tube that is wrapped around the barrel and that is coupled to the housing so that it remains fixed within the housing as the barrel rotates;
a carriage track that is coupled to the barrel so that the carriage track rotates around the rotational axis as the barrel rotates, wherein the carriage track extends along the barrel in a direction that is parallel to the rotational axis;
a carriage that is movable along the carriage track and simultaneously along the guide tube; and
a rotator that is housed within the carriage so that the rotator is rotatable about an axis that is tangent to a direction of movement of the carriage, wherein the rotator is coupled to the guidewire so that as the carriage moves, the guidewire advances through the guide tube.

3. (canceled)

4. The cartridge of claim 1, wherein the guide tube defines a helical notch.

5. The cartridge of claim 1, wherein the shield has a quarter toroid shape.

6. The cartridge of claim 1, further comprising at least one encoder that is configured to measure an angular displacement of the barrel.

7. (canceled)

8. The cartridge of claim 1, further comprising a plurality of rollers that rotatably support an interior of the barrel.

9. (canceled)

10. The cartridge of claim 1, further comprising a dummy source wire.

11. The cartridge of claim 1, wherein at least a portion of the shield is removable and replaceable.

12. The cartridge of claim 1, further comprising a loading port, wherein at least a portion of the loading port is removable and replaceable.

13. (canceled)

14. The cartridge of claim 2, wherein the guide tube is a first guide tube, wherein the cartridge is configured to receive a second guide tube in place of the first guide tube, wherein the second guide tube differs from the first guide tube in pitch.

15. The cartridge of claim 10, further comprising a drive and control system that is configured to extend the dummy wire from the cartridge.

16. A system comprising:
a cartridge having:
a radiation source;
a shield that is configured to receive the radiation source;
a guidewire coupled to the radiation source; and
at least one motor that is configured to move the guidewire to thereby adjust the position of the radiation source; and
a base having:
a receptacle that is configured to receive the cartridge, wherein the receptacle defines a plurality of contacts for providing power and control signals to the cartridge; and
a controller that is configured to communicate with the cartridge via the plurality of contacts.

17. (canceled)

18. (canceled)

19. (canceled)

20. (canceled)

21. (canceled)

22. The system of claim 16, further comprising:
at least one radiation detector; and
a controller that is operable:
to receive signals from the at least one radiation detector; and
determine, based on the signals received from the at least one radiation detector, if the radiation source has been extended or retracted from the cartridge.

23. The system of claim 22, wherein the at least one radiation detector is in the cartridge.

24. (canceled)

25. The system of claim 16, further comprising a controller that is configured to determine a current measurement through the at least one motor and, based on the current measurement, if the motor current has exceeded a first threshold.

26. The system of claim 25, wherein the controller is configured to determine whether the current has exceeded a second threshold, wherein the first threshold is associated with rotation in a first direction, and the second threshold is associated with rotation in a second direction that is opposite the first direction.

27. (canceled)

28. The system of claim 16, further comprising a controller that is configured to detect an error in operation.

29. The system of claim 28, wherein the controller is configured to cause the cartridge to retract the active source in response to detecting the error in operation.

30. (canceled)

31. (canceled)

32. (canceled)

33. (canceled)

34. A method comprising:
inserting a first cartridge into a receptacle of an afterloader base, wherein the first cartridge comprises an active source.

35. The method of claim 34, further comprising:
removing the first cartridge from the receptacle of the afterloader base; and
inserting a second cartridge into the receptacle of the afterloader base.
